(19) United States
(12) Patent Application Publication
(10) Pub. No.: US 2013/0137928 A1
(43) Pub. Date: May 30, 2013

(54) ENDOSCOPE

(71) Applicant: FUJIFILM CORPORATION, Tokyo (JP)

(72) Inventors: Hiroyuki KARASAWA, Ashigara-kami-gun (JP); Masahiko NUMANO, Ashigara-kami-gun (JP)

(73) Assignee: FUJIFILM CORPORATION, Tokyo (JP)

(21) Appl. No.: 13/686,671

(22) Filed: Nov. 27, 2012

(30) Foreign Application Priority Data
Nov. 28, 2011 (JP) 2011-258745

(51) Int. Cl. A61B 1/005; A61B 1/267

(52) U.S. Cl. CPC A61B 1/0057; A61B 1/2676
USPC 600/149

(57) ABSTRACT

An endoscope permitting cleaning thereof with hydrogen peroxide and having an angle section with enhanced ease of operation. The endoscope includes the angle section including substantially cylindrical tubular members connected to each other, a bending operation section for bending the angle section, and wires for connecting the bending operation section and the angle section and bending the angle section when drawn by the operation at the operating section. The wires have adhered on the surfaces thereof polytetrafluoroethylene powder produced by pyrolysis as lubricant.

### BACKGROUND OF THE INVENTION

The present invention relates to an endoscope. More particularly, the present invention relates to an endoscope permitting cleaning thereof with hydrogen peroxide and having an angle section providing enhanced ease of operation.

As is known, endoscopes are inserted into a biological body such as a human body for examination and diagnosis of organs and the like.

An endoscope basically includes an insertion section that is inserted into a human body, an operating section for manipulating the insertion section and performing other operations of the endoscope such as air/water supply, a connector (LG (light guide) connector) connected to, for example, an air supply source and a suction pump, and a universal cord (LG flexible section) for connecting the connector to the operating section and the insertion section.

The insertion section comprises a tip portion incorporating an imaging unit including a CCD sensor and an illumination lens and an elongate flexible section on the proximal side.

Between the tip portion and the flexible section of the insertion section, there is typically provided an angle section (bending section) that bends upwards and downwards or, optionally, in the left and right directions (directions (substantially) perpendicular to the upward and downward directions).

As is known, the angle section is typically composed of a plurality of substantially cylindrical tubular members (articular rings/(angle) rings) connected so as to pivot in the directions in which the angle section is bent.

Such angle section is bent typically by drawing a wire (angle wire) connecting the operating section and the angle section.

Specifically, an endoscope comprises a manipulative knob rotatably provided on the operating section, a pulley turned by the rotation of the manipulative knob, and wires each having one end thereof secured to the pulley and the other end connected to a tubular member located at the distal end of the angle section. The wires are provided apart in two directions in which the angle section is bent and secured to the pulley so as to be drawn on one side and forwarded on the other side as the manipulative knob is turned.

Accordingly, rotation of the manipulative knob causes one of the wires to be drawn so that the angle section composed of connected tubular members is bent toward the side on which the wire is drawn.

Needless to say, it is preferable that the angle section can be bent with a small force. Therefore, the surfaces of the wires for bending the angle section are coated with lubricant. In addition to the wires, consideration needs to be taken for the tube constituting the forceps channel, the light guide, and other components that come into sliding contact with each other when, for example, the insertion section is bent when inserted into a body cavity, in order to prevent damage that may be thereby caused to such components. Therefore, lubricant is also applied onto the surfaces of such components contained in the insertion section.

The lubricant used for such purpose is in most cases molybdenum disulfide.

To prevent, for example, transmission of infectious diseases, cleaning and a high-level disinfection of endoscopes are recommended after every use thereof. As regards bronchoscopes, in particular, sterilization thereof is desirable.

Among highly effective sterilization methods is known a method using hydrogen peroxide. Low-temperature plasma sterilization method using hydrogen peroxide is known to permit low-temperature treatment, be quick, and produce extremely high sterilizing effects.

However, when the lubricant used is molybdenum disulfide, sterilization of the endoscope using hydrogen peroxide causes molybdenum disulfide to react with hydrogen peroxide, leading to generation of sulfides such as sulfuric acid, which damages the endoscope and causes malfunction thereof (see JP 11-28184 A, JP 2004-208962 A and JP 2006-81749 A).

### SUMMARY OF THE INVENTION

Therefore, JP 11-28184 A proposes using, for example, carbon graphite, boron nitride, polytetrafluoroethylene, fluorinated oil, fluorinated grease instead of molybdenum disulfide as lubricant for endoscopes.

JP 2004-208962 A proposes using as lubricant for endoscopes a composition containing 3 to 20 wt % of a given component selected from, for example, fluoropolymer resin and polyimide, 10 to 40 wt % of epoxy resin and phenol resin as binder, 0.05 to 10 wt % of dispersant, and 30 to 85 wt % of organic solvent.

JP 2006-81749 A proposes using powder of porous carbonaceous material as lubricant for endoscopes.

However, such conventional lubricants often fail to produce sufficient lubricating effects as lubricant for the wires for bending the angle section. Thus, a great force is often still required to bend the angle section (turn the manipulative knob) when any of such lubricants is applied to the wires.

In addition, while such lubricants used on the wires may initially produce good lubricating effects, repeated use and repeated cleaning, disinfection, and sterilization gradually reduce the lubricating effects. Accordingly, a great force becomes necessary as time passes to bend the angle section.

While using carbon graphite, boron nitride, or polytetrafluoroethylene as solid lubricant in place of molybdenum disulfide is known as described above, their appropriate particle shapes, sizes, and the like as lubricant are not as yet made clear.

Because polytetrafluoroethylene, in particular, is not inorganic material but polymeric material, the particle shapes and sizes alone do not determine the performance as lubricant. In other words, the properties of polytetrafluoroethylene as lubricant may greatly depend on, for example, the synthesis method, the polymerization conditions, and the purification method employed. However, when dealing with, in particular, endoscopes requiring repeated low-temperature plasma sterilization using hydrogen peroxide, not only were the method and conditions for producing polytetrafluoroethylene capable of restricting the change in operation of bending the angle section unknown, but it was also not known that the properties of polytetrafluoroethylene as solid lubricant are affected by the synthesis method, the polymerization conditions, and the purification method.

The present invention has an object of solving the above problems in the art and providing an endoscope permitting sterilization using hydrogen peroxide and exhibiting enhanced ease of operation in bending the angle section over an extended period of time. The present invention provides, in particular, an endoscope using polytetrafluoroethylene, a polymeric material, as solid lubricant such that the properties thereof do not change with frequent use of the endoscope and repeated cleaning, disinfection, and sterilization.

To achieve the above object, the endoscope of the present invention comprises an angle section close to a distal end of an insertion section, the endoscope further comprising a bending operation section for bending the angle section and wires for connecting the bending operation section and the angle section, the wires being drawn by operation of the bending operation section to bend the angle section, the angle section including substantially cylindrical tubular members connected in a longitudinal direction of the insertion section, the wires having adhered on surfaces thereof polytetrafluoroethylene powder produced by pyrolysis as lubricant.

It is preferable that the tubular members each have a projection formed by forming two cuts in a peripheral surface thereof arranged in a direction in which the insertion section extends and pressing a portion between the two cuts so as to bulge on an inside of each of the tubular members, and the wires are passed through the projections.

The tubular members preferably have an inner diameter of 5 mm or less.

Powder of the polytetrafluoroethylene preferably has a particle diameter of 5 to 50 μm.

Powder of the polytetrafluoroethylene preferably adheres to the wires in an amount of 0.1 to 2 μg/cm.

The wires are preferably twisted wires.

Powder of the polytetrafluoroethylene preferably permeates between lines constituting the twisted wires.

The endoscope is preferably an endoscope for bronchi.

The endoscope of the invention uses powder of polytetrafluoroethylene (hereinafter referred to also as PTFE) produced by pyrolysis as lubricant for coating the wire used to bend the angle section.

As shown also in JP 11-28184 A above, PTFE is not decomposed in the sterilization process of endoscopes using hydrogen peroxide. Moreover, PTFE obtained by pyrolysis excels not only in lubricity but causes only a small change in angle torque through continued and frequent use, and degrades little through cleaning, disinfection, and sterilization performed after each use of the endoscope.

Therefore, the endoscope of the invention permits sterilization to be performed using hydrogen peroxide such as low-temperature plasma sterilization using hydrogen peroxide capable of high sterilization efficiency. Further, the present invention provides an endoscope enabling the angle section to be bent with a small force and thereby providing enhanced ease of operation over an extended period of time.

### BRIEF DESCRIPTION OF THE DRAWINGS

FIG. 1 is a conceptual view illustrating an example of the endoscope of the invention.

FIGS. 2A to 2D are conceptual views for explaining the angle section of the endoscope illustrated in FIG. 1.

FIG. 3 is a conceptual view for explaining another example of the angle section.

FIG. 4 is a conceptual view for explaining the angle section of the endoscope illustrated in FIG. 1.

FIG. 5 is a graph illustrating a relation between the angle of the angle section and the pull strength.

FIG. 6 is a graph illustrating a relation between sterilization and the angle torque of the angle section.

FIG. 7 is a graph illustrating a relation between sterilization and the angle torque of the angle section.

FIG. 8A is a graph illustrating a relation between the number of times an endurance test was conducted and the angle torque of the angle section; FIG. 8B is a graph illustrating a rate of change in the angle torque in FIG. 8A.

### DETAILED DESCRIPTION OF THE INVENTION

Hereinafter, the endoscope of the present invention is described in detail with reference to the preferred embodiments illustrated in the accompanying drawings.

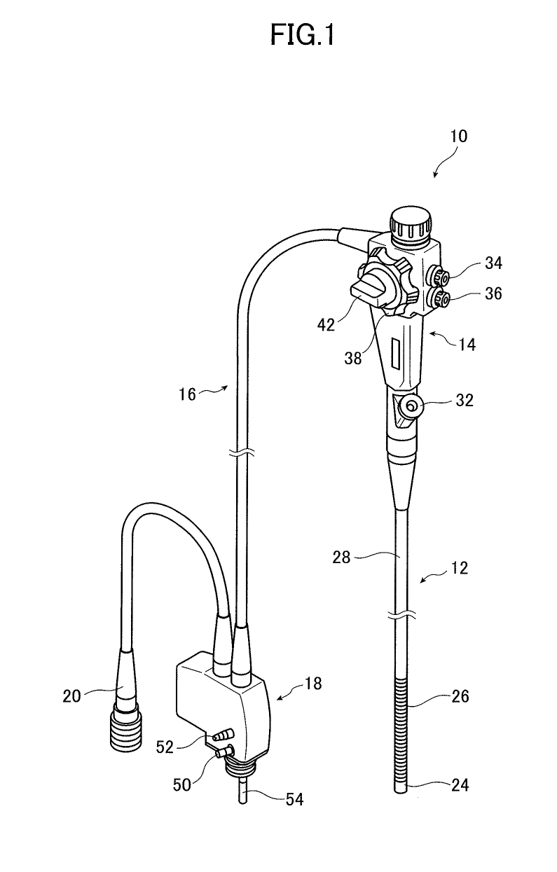

FIG. 1 conceptually shows an example of the endoscope of the invention.

An endoscope 10 illustrated in FIG. 1 is inserted into a site of a body such as a body cavity (e.g., bronchi and stomach) to be treated or examined for observation of the inside of the body, acquisition of still images and moving images of the inside of the body, collection of biological tissues, and other purposes.

The endoscope 10 is a so-called electronic scope type endoscope using a CCD sensor to acquire still and moving images of a site to be examined for observation. Like typical endoscopes, the endoscope 10 includes an insertion section 12, an operating section 14, a universal cord 16, an LG connector 18, and a video connector 20.

The endoscope 10 of the invention can be appropriately used for examination and diagnosis of a variety of sites including bronchi, pharynx, and alimentary canal including the duodenum and for surgery.

As described in detail later, the endoscope 10 of the invention uses polytetrafluoroethylene (PTFE) produced by pyrolysis as lubricant for wires 68 for bending an angle section 26 of the insertion section 12. Thus, the endoscope 10 of the invention permits sterilization using hydrogen peroxide capable of enhanced sterilization. Further, the endoscope 10 of the invention maintains enhanced ease of operation in bending the angle section 26 over an extended period of time even with the insertion section 12 having a reduced diameter.

Thus, the present invention can be particularly suitably applied to bronchoscopes that require a high level of sterilization and of which the insertion section 12 is required to have a reduced diameter.

The insertion section 12 is an elongate part that is inserted into a site to be examined such as a body cavity and, like conventional endoscopes, has a tip portion 24 at the distal end (the leading end of the insertion section being the opposite end from the operating section 14), the angle section 26, and a flexible section 28. The endoscope 10 of the invention is characterized by wires 68 for bending the angle section 26 or a lubricant therefor and otherwise is a known endoscope.

The operating section 14 is provided to operate the endoscope 10.

As of a typical endoscope, the operating section 14 comprises a forceps inlet 32 communicating with a forceps aperture at the tip portion 24 of the insertion section 12 through a forceps channel for inserting a treatment tool such as forceps, a suction button 34 for causing suction through the forceps aperture at the tip portion 24, and an air/water supply button 36 for supplying air and water from a nozzle at the tip portion 24 through an air/water supply channel.

Although the illustrated example of the endoscope 10 has a suction function and an air/water supply function, the endoscope of the invention may have only one or none of the suction function and the air/water supply function. Alternatively, the endoscope 10 may include other functions in place of or in addition to the suction function and/or the air/water supply function.

The operating section 14 further comprises a UD knob 38 for bending the angle section 26 upwards and downwards (in a given direction and in the opposite direction from the given direction) and a UD brake 42 for keeping the angle section 26 bent.

The operating section 14 also incorporates a pulley 82 located coaxially with the UD knob 38 inside the operating section 14 and rotated with the UD knob 38. Over the pulley 82 are passed the wires 68 for bending the angle section 26. This will be described in detail later.

The operating section 14 of the endoscope 10 further comprises switches for observing and acquiring images with an imaging unit (CCD sensor) including a zoom switch, a still image shooting switch, a moving image shooting switch, and a freeze switch.

The LG (light guide) connector 18 is a section for connecting the endoscope 10 to a water supply means, an air supply means, a suction means, and the like of the facilities where the endoscope 10 is used.

In the example shown, the LG connector 18 comprises a water supply connector 50 for connecting the endoscope 10 to the water supply means of the facilities, an air supply connector 52 for connecting the endoscope 10 to the air supply means of the facilities, and a suction connector for connecting the endoscope 10 to the suction means of the facilities. The LG connector 18 further comprises an LG rod 54 for connection to an illumination light source and an S terminal provided for connection to an S cord when an electronic knife is used.

Because the endoscope 10 is an electronic scope as earlier mentioned, the LG connector 18 is connected to the video connector 20 for connecting the endoscope 10 to a processor.

Images (image data) acquired by the imaging unit (CCD sensor) provided in the tip portion 24 and instructions given from the operating section 14 are transmitted through signal lines via the LG connector 18 and from the video connector 20 on to the processor.

The universal cord (LG flexible section) 16 connects the LG connector 18 to the operating section 14.

The universal cord 16 has therein housed/passed a water supply channel connected to the water supply connector 50, an air supply channel connected to the air supply connector 52, a suction channel connected to the suction connector, a light guide for delivering observation light emitted from the illumination light source to the LG rod 54, and the signal lines for transmitting instructions given using the operating section 14 and image data acquired by the imaging unit of the tip portion 24.

The suction channel is connected through the suction button 34 to the forceps channel communicating with the forceps aperture at the tip portion 24 of the insertion section 12. The water supply channel and the air supply channel are connected through the air/water supply button 36 to the air/water supply channel communicating with the nozzle at the tip portion 24 described earlier. The light guide is passed through the operating section 14 to the illumination lens at the tip portion 24. As described above, the signal lines are passed from the video connector 20 through the LG connector 18 and the operating section 14 to the imaging unit at the tip portion 24.

The insertion section 12 of the endoscope 10 comprises, as earlier mentioned, the tip portion 24, the angle section 26, and the flexible section 28.

The tip portion 24 located at the distal end of the insertion section 12 incorporates the imaging unit where the CCD sensor, the imaging lens, and the like are integrally provided, the illumination lens for illuminating a site to be observed with observation light transmitted by the light guide, and other components. The tip portion 24 further comprises the forceps aperture for inserting the forceps into a site to be treated and the nozzle for supplying air and water.

The angle section (bending section) 26 is a region that can be bent upwards and downwards by operating the UD knob 38 provided at the operating section 14 in order to insert the tip portion 24 to a target position or locate it in a target position. The angle section 26 will be described in detail later.

The flexible section 28 connects the tip portion 24 and the angle section 26 to the operating section 14 and is an elongate tube having sufficient flexibility to permit insertion into a site to be examined.

As described earlier, through the flexible section 28 and the angle section 26 are passed the forceps channel (suction channel) for inserting the forceps into a site under treatment, the air/water supply channel (air supply channel and water supply channel) for supplying air and water in response to the operation of the air/water supply button 36, the signal lines for transmitting image data acquired by the imaging unit (CCD sensor) at the tip portion 24, and the light guide for transmitting illumination light, among other components.

The flexible section 28 and the angle section 26 have also passed therethrough the wires 68 for bending the angle section 26, as described later.

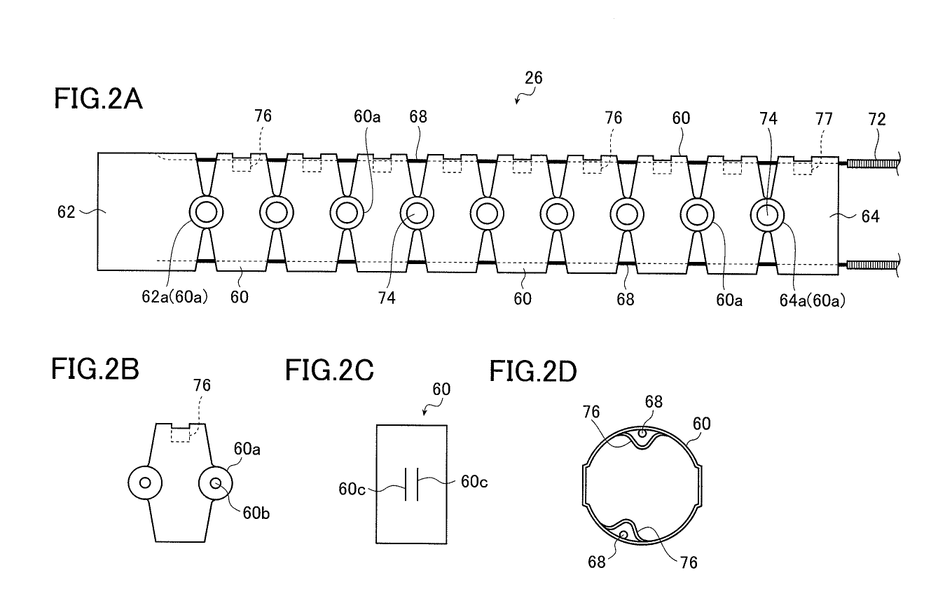

FIG. 2A illustrates the concept of a configuration of the angle section 26.

Like the angle sections of various endoscopes, the angle section 26 is configured by connecting a plurality of substantially cylindrical rings (articular rings), inserting wires (angle wires) through the rings for bending the angle section 26, and securing the wires to a ring at the distal end.

The angle section 26 in the illustrated example comprises a total of 10 substantially cylindrical rings (tubular members) connected to each other: eight rings 60, a distal ring 62 at the distal end, and a proximal ring 64 at the proximal end (closer to the operating section 14).

The proximal ring 64 and the rings 60 have passed therethrough two wires 68 for bending the angle section 26. The ends of the wires 68 at the distal end are secured to the distal ring 62. The other ends of the wires 68 at the proximal end are passed over the pulley 82 of the operating section 14 described later.

The sections of the wires 68 from positions somewhat away from the pulley 82 (or, for example, a chain, described later) and as far as slightly short of the angle section 26 are passed through coils 72 (springs) each composed of an extremely thin, spirally winding wire.

Each ring 60 has a shape as conceptually illustrated in FIG. 2B such that both ends of the cylinder in the axial direction are cut diagonally so that the axial length of the cylinder gradually decreases from apexes, or positions at which a certain diameter passes through the cylinder, in a direction perpendicular to that diameter. Thus, both ends of each ring 60 have chevron-like shapes with apexes at which diameters extending in the same direction pass the ring.

At the apexes (summits of the chevrons) passed through by the diameters, engagement members 60a each having the shape of a circle (disk) with the center located at the apex are provided. The engagement members 60a each have a through-hole 60b at the center.

The distal ring 62 is a substantially cylindrical member longer than the rings 60 and having a flat end on one side and a chevron-shaped end as of the rings 60 on the other side. The distal ring 62 has circular engagement members 62a provided at the summits of the chevrons similar to the engagement members 60a. The engagement members 62a likewise have a through-hole, not shown, at the center thereof.

The proximal ring 64 is also a substantially cylindrical member slightly longer than the rings 60 and having a flat end on one side and a chevron-shaped end as of the rings 60 on the other side. The proximal ring 64 has circular engagement members 64a provided at the summits of the chevrons similar to the engagement members 60a. The engagement members 64a likewise have a through-hole, not shown, at the center thereof.

The eight rings 60 are arranged with the through-holes 60b of one ring 60 aligned with those of an adjacent ring or rings 60 and connected by rivet-shaped link members 74 passed through through-holes 60b so that the rings 60 may pivot about the axes provided by the link members 74 with respect to each other.

The distal ring 62 is arranged with its through-holes in the engagement members 62a aligned with the through-holes 60b of the foremost ring 60 and connected, as are the rings 60, by the link members 74 with the foremost ring 60 so as to be pivotable with respect to the foremost ring 60. The proximal ring 64 is arranged with its through-holes formed in the engagement members 64a aligned with the through-holes 60b of the rearmost ring 60 and connected, as are the rings 60, by the link members 74 with the rearmost ring 60 so as to be pivotable with respect to each other.

As conceptually illustrated in FIG. 2C, the rings 60 each have in the peripheral surface (lateral surface) two cuts 60c so as to be apart from each other in the axial direction of the rings 60 (direction in which the insertion section extends) and in the same position as seen in the direction perpendicular to the axial direction (referred to also as circumferential direction below). Each cut 60c extends in the circumferential direction. In the illustrated example, the cuts 60c are formed in the same position as seen in the circumferential direction and extend in the circumferential direction in a preferred embodiment, but the present invention is not limited thereto.

In each ring 60, the portion between the cuts 60c is pressed toward the center of the ring 60 (cylinder) so as to bulge inwardly. Thus, each bulge forms a tubular projection 76 with the inner peripheral surface of the ring 60, as conceptually illustrated in FIG. 2D where a ring 60 is seen from the axial direction. The projections 76 of the rings 60 are formed through so-called cutting and drawing process to provide tubular portions extending in the axial direction. Thus, portions of the peripheral surface of each of the rings 60 are formed into passages for the respective wires, constituting wire passages integral with each of the rings.

In the example illustrated in FIG. 2D, each ring 60 has two such projections 76, one in a position corresponding to one end of a diameter perpendicular to a line connecting the through-holes 60b in the engagement members 62a (i.e., the apexes described earlier) and the other in a position slightly shifted in the circumferential direction from a position corresponding to the other end of that diameter.

However, the positions of the projections 76 are not limited to those in the illustrated example; for example, two projections 76 may be located both on the same diameter.

In the angle section 26, the proximal ring 64 has similar projections 77 formed by cutting and drawing process in the same positions in the circumferential direction as in the rings 60.

As illustrated in FIGS. 2A and 2D, the wires 68 are passed through the projections 76 and the projections 77 from the proximal ring 64 linearly through the eight rings 60, and the distal ends of the wires 68 are secured to the inner wall surface of the distal ring 62.

Thus, in a preferred embodiment of the angle section 26, the rings 60 and the proximal ring 64 are subjected to cutting and drawing process to form passages allowing the wires 68 to pass through given positions of the rings.

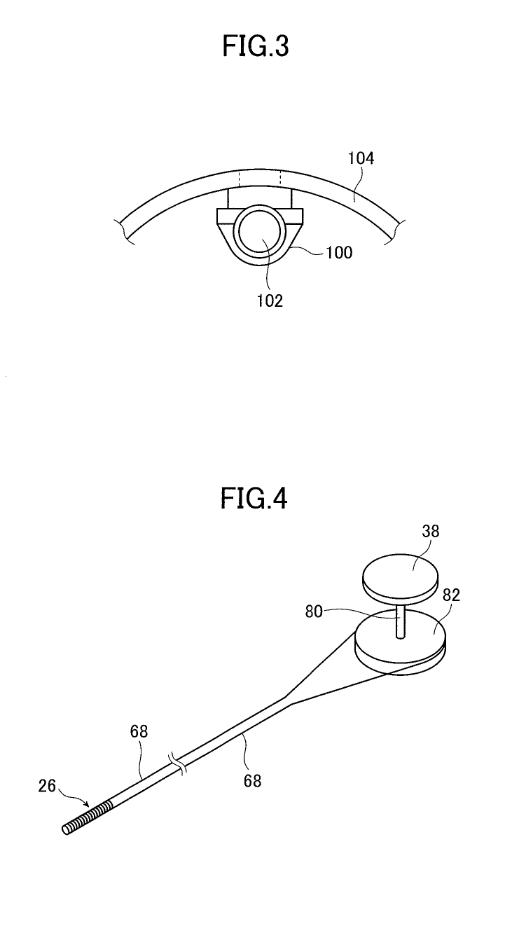

In general, the wire passages in the angle section of endoscopes are provided using pin-like members 100 having a through-hole 102 for passing a wire therethrough and fitted into through-holes formed in the peripheral surfaces of (angle) rings 104 as conceptually illustrated in FIG. 3.

However, the pin-like members 100 project significantly on the inside of the rings 104 and thus reduce the cross sectional area of the rings 104. Should the pin-like members 100, therefore, be used to provide wire passages in the angle section of bronchoscopes and hence required to have an insertion section having a reduced diameter, the insertion section would be unable to allow necessary components such as the forceps channel and the light guide to be passed therethrough.

By contrast, the structure as illustrated in FIG. 2 with the wires 68 passed through the projections 76 formed in the peripheral surfaces of the rings 60 by cutting and drawing process provides passages for the wires 68 in the angle section 26 without the need to form portions that project significantly on the inside of the rings 60.

Therefore, endoscopes having the insertion section 12 with a reduced diameter can still contain necessary components. Thus, passages for appropriately passing the wires 68 formed in the rings 60 and the like constituting the angle section 26 through cutting and drawing process allow the insertion section 12 to have a reduced diameter so that the endoscope can be suitably used also for applications permitting only a thin insertion section 12 to be used as for bronchi.

According to the present invention, the passages for the wires 68 in the angle section 26 are not however limited to the projections 76 formed by cutting and drawing process as in the illustrated preferred embodiment of the endoscope 10 for reducing the diameter of the insertion section 12.

Specifically, the passages for the wires 68 in the angle section 26 may be of any configuration used for known endoscopes including the pin-like member 100 illustrated in FIG. 3.

The inner diameters of the rings 60, the distal ring 62, and the proximal ring 64 constituting the angle section 26 are not specifically limited and may be determined as appropriate according to the applications for which the endoscope 10 is used.

In whatever application the endoscope 10 may be used, the insertion section 12 of the endoscope 10 preferably has as small a diameter as possible in order to reduce strain on patients. In particular, bronchoscopes are required to have a thin insertion section 12. Where the passages for the wires 68 in the angle section 26 are formed through the above-mentioned cutting and drawing process that is preferable for reducing the diameter of the insertion section 12, the effect of reducing the diameter is especially significant as described later.

To better produce the effects of the invention and for other advantages, the inner diameters of the rings 60, the distal ring 62, and the proximal ring 64 constituting the angle section 26 are preferably 5 mm or less, and in particular, 4 mm or less.

In the endoscope 10 of the invention, the wires 68 are not specifically limited and may be any of known wires used in endoscopes to bend the angle section such as wires made by twisting metal lines, wires made by twisting a plurality of twisted metal wires, and a single metal wire.

Preferably, the wires 68 of the endoscope 10 of the invention are wires each composed of not a single line but a twisted wire (a wire like a twisted yarn).

The diameter of the wires 68 is not specifically limited and may be determined as appropriate according to, for example, the kind of the endoscope, the thickness of the insertion section 12, and components housed in the insertion section 12.

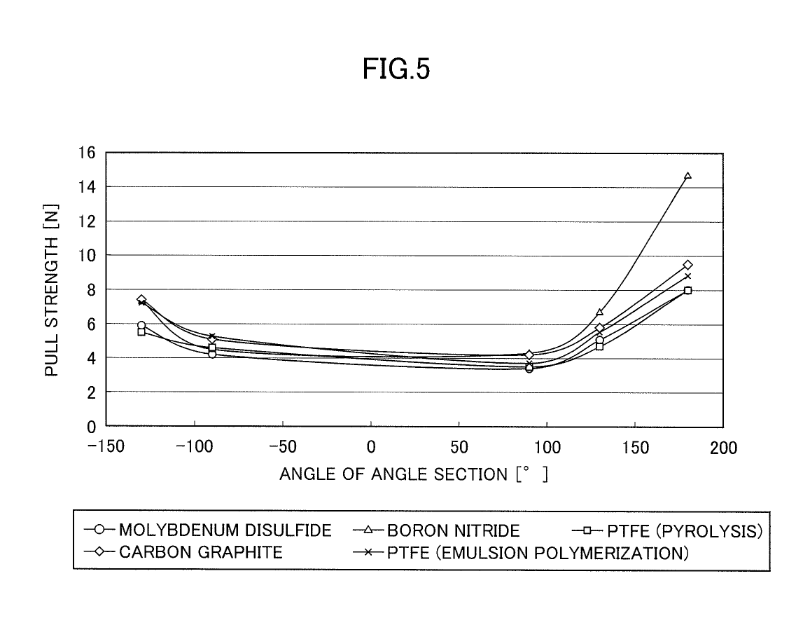

FIG. 4 illustrates a concept of a mechanism for bending the angle section 26.

The UD knob 38 is secured to a rotary shaft 80 so supported as to be rotated by the operating section 14. Thus, the UD knob 38 is rotatably supported by the rotary shaft 80.

To the lower end of the rotary shaft 80, the pulley 82 is secured concentrically with the rotary shaft 80. Thus, the pulley 82 turns in the same direction as the UD knob 38 upon rotation of the UD knob 38.

The pulley 82 engages and has wound thereon the proximal ends of the two wires 68 that are passed through the proximal ring 64 and the eight rings 60 and of which the distal ends are secured to the distal ring 62. One of the wires 68 is wound on the pulley 82 clockwise; the other wire 68 is wound counterclockwise on the pulley 68.

Thus, rotation of the UD knob 38 causes the pulley 82 to turn in the same direction so that one of the wires 68 is wound and drawn, and the other is reeled out from the pulley 82.

As described above, the rings 60 and other components of the angle section 26 are pivotally connected by the link members 74 at engagement members 60a (through-holes 60b) lying on diameters. The two wires 68 are passed through the projections 76 and the like formed by cutting and drawing process, each located in a position lying on a diameter perpendicular to the line connecting the engagement members 60a and in a position slightly shifted from the diameter.

Thus, as described above, when the UD knob 38 turns and the pulley 82 thereby turns, drawing one of the wires 68 while reeling out the other wire 68, the angle section 26 bends towards the side on which one of the wires 68 is drawn by an amount by which the wire is drawn.

According to the invention, the number of wires 68 is not limited to two; one wire may be passed over and wound on the pulley 82 in a U-shape and, as in the above-mentioned configuration, through the angle section 26, with the ends secured to the distal ring 62.

The invention is not limited to the configuration wherein the wires 68 passed through the angle section 26 are directly wound on the pulley 82. For example, a length of chain may be wound on a pulley having the form of a sprocket gear, with both ends of the chain connected to the respective ends of the wires 68. Further, there may be provided a connection member between the chain and each of the wires 68 to connect them.

Although the angle section 26 is bent only in two directions, upwards and downwards (substantially in opposite directions), in the illustrated example of the endoscope 10, the endoscope of the invention is not limited in this manner.

The endoscope of the invention may have an angle section capable of bending in, for example, four directions, i.e., in the up- and downward directions and in two additional directions, left- and rightward directions substantially perpendicular to the up and downward directions, as do the endoscopes for digestive organs.

Thus, the angle section 26 of the endoscope of the invention may have any configuration as appropriate used in known endoscopes including the configuration of the operating section 14 for bending such as the wires 68, the coils 72, and the pulley 82.

The wires 68 for bending the angle section 26 of the endoscope 10 of the invention have adhered on the surfaces thereof powder of polytetrafluoroethylene (hereinafter referred to also as PTFE) produced by pyrolysis as lubricant.

Pyrolysis is a method of producing powder of PTFE whereby high-molecular weight PTFE is thermally decomposed in inert atmosphere and thus reduced to low molecular weight PTFE, which is then pulverized to obtain PTFE in the form of fine particles.

As described earlier, the surfaces of the components, such as the wires and the light guide, housed in the insertion section of the endoscope are coated with a lubricant as described earlier for the purpose of improving the ease of bending of the angle section and protecting the components as the insertion section is inserted into a body cavity. Much use is made of molybdenum disulfide as lubricant for endoscopes.

Endoscopes are preferably cleaned, disinfected, and sterilized after each use thereof. Among sterilization methods known to produce high sterilizing effects are ones employing hydrogen peroxide such as low-temperature plasma sterilization using hydrogen peroxide.

However, when sterilization using hydrogen peroxide is performed for an endoscope that uses molybdenum disulfide as lubricant, molybdenum disulfide decomposes, generating sulfides such as sulfuric acid, which damages the endoscope and causes malfunction thereof.

Lubricants permitting sterilization using hydrogen peroxide are exemplified by carbon graphite powder, boron nitride powder, fluorinated oil, and fluorinated grease.

However, these lubricants often fail to produce sufficient lubricating effects as lubricant for the wires 68 for bending the angle section 26 and often fail to provide sufficient ease of operation in bending the angle section. In addition, despite initially good lubricating effects, many of these lubricants gradually lose their lubricating performance through repeated use or cleaning, allowing the ease of operation in bending the angle section to decrease with time in many of the cases.

PTFE is another example of lubricant that may be used when the sterilization uses hydrogen peroxide. PTFE is a polymeric material and produced using a variety of synthesis methods, polymerizing conditions, and purification methods. However, no discussion has been hitherto made as to which methods and which conditions are appropriate for applications where PTFE is used as lubricant for endoscopes. Also known, besides PTFE powder produced by pyrolysis, is PTFE powder produced by emulsion polymerization (direct polymerization) method. By emulsion polymerization method, PTFE dispersion produced by emulsion polymerization is agglomerated and dried to obtain PTFE powder.

However, PTFE powder obtained by the emulsion polymerization method produces lower lubricating effects as lubricant for the wires 68 for bending the angle section 26 than PTFE powder obtained by pyrolysis. Moreover, the PTFE powder obtained by the emulsion polymerization method degrades more greatly through, for example, use and sterilization as time passes. In addition, continued use of PTFE powder obtained by the emulsion polymerization method changes the angle torque and thereby leads to inconsistent operation.

To enhance the ease of operation in bending the angle section 26, good slidability needs to be secured between the wires 68 and the angle section 26 (e.g., rings 60).

PTFE powder obtained by pyrolysis has an enhanced lubricity and can increase the slidability. Further, the PTFE powder obtained by pyrolysis not only has an enhanced lubricity but exhibits high particle configuration stability. In addition, because even PTFE powder of dry type obtained by pyrolysis has good dispersibility, it can be adhered uniformly over the whole surface of the wires 68.

Therefore, the endoscope 10 using PTFE powder obtained by pyrolysis as lubricant for the wires 68 for bending the angle section 26 permits bending operation to be effected with a small force and enables enhanced ease of operation in bending the angle section 26.

Further, the lubricant applied to the wires 68 receives localized forces as the wires 68 slide over the angle section 26 (e.g., rings 60). Accordingly, the lubricant for the wires 68 is required to have a high particle configuration stability free from crushing, pulverization, and the like in order to secure enhanced ease of operation in bending the angle section 26 over an extended period of time.

PTFE powder obtained by pyrolysis has a high particle configuration stability as described above, and this also applies where sterilization uses hydrogen peroxide. It follows therefore that the endoscope 10 of the invention using the PTFE powder obtained by pyrolysis as lubricant for the wires 68 for bending the angle section 26 provides an angle torque that changes little when used frequently for a long time, and can maintain enhanced ease of operation in bending the angle section 26 consistently over an extended period of time.

As described above, the condition of the material being PTFE alone does not enable a lubricant for endoscopes to have appropriate properties; desired results can only be obtained when the production method is also specified.

The lubricating effects produced by the use of PTFE powder obtained by pyrolysis as lubricant are especially great for the endoscope 10 having the angle section 26 in which the wires 68 are passed through the projections 76 and the projections 77 formed by cutting and drawing process and which thereby enables reduction in diameter of the insertion section 12.

Where the angle section uses the pin-like members 100 having the through-hole 102 for passing the wire as illustrated in FIG. 3, the wire basically comes into contact only with the through-holes 102 of the pin-like members 100 as the angle section bends. It virtually means that the wire only comes into point contact with the other members in the angle section.

By contrast, the projections 76 formed by cutting and drawing process provide tubular passages for passing the wires 68 therethrough together with the inner peripheral surfaces of the rings 60. Likewise, the projections 77 formed by cutting and drawing process provide tubular passages for passing the wires 68 therethrough together with the inner peripheral surfaces of the rings 64.

Thus, when the angle section 26 is bent, the wires 68 come into contact with the inner peripheral surfaces of the rings 60 and the like substantially throughout the length. Accordingly, with the configuration formed by using cutting and drawing process, the area with which the wires 68 come into contact with the rings 60 and the proximal ring 64 is large and, hence, the sliding resistance and the friction force are also great.

Therefore, in the angle section 26 where the wires 68 are passed through the projections 76 and other members formed by cutting and drawing process, the effects produced by the lubricant for the wires 68 on the bending operation are extremely great as compared with the case using the pin-like members 100 as illustrated in FIG. 3.

However, as described above, the PTFE powder produced by pyrolysis has not only excellent lubricity but a highly stable particle configuration, and this applies even where sterilization uses hydrogen peroxide.

Therefore, even where the angle section 26 has the configuration where the wires 68 are passed through the projections 76 and other components formed by cutting and drawing process as in the illustrated example of the endoscope 10, use of PTFE powder obtained by pyrolysis as lubricant for the wires 68 enables enhanced ease of operation in bending the angle section 26 to be maintained over an extended period of time.

FIG. 5 illustrates a relation between the angle [°] of the angle section and the pull strength [N] on the wire required to bend the angle section to that angle using various lubricants for the wires for bending the angle section of an endoscope produced by FUJIFILM Corporation. The pull strength herein is a pull strength applied to the wire for bending the angle section of an endoscope when the wire is directly pulled to bend the angle section.

The lubricants used in this example were PTFE powder obtained by pyrolysis, PTFE powder obtained by emulsion polymerization, molybdenum disulfide powder, boron nitride powder, and carbon graphite powder.

As illustrated in FIG. 5, use of PTFE powder obtained by pyrolysis as lubricant for the wires enables the angle section of the endoscope to be bent with a pull strength equivalent to that required when molybdenum disulfide powder, which is conventionally used as lubricant, is used as lubricant for the wires, without regard to the angle of the angle section.

Further, the initial angle torque [N·cm] and angle torques after sterilization were measured using an endoscopes produced by FUJIFILM Corporation, where PTFE powder obtained by different production methods was used as lubricant for the wires for bending the angle section.

The angle torque herein is a turning force applied to the UD knob when the UD knob is operated to bend the angle section of the endoscope.

Two kinds of PTFE powder were used: PTFE powder obtained by pyrolysis, and PTFE powder obtained by emulsion polymerization.

Angle torques were obtained by measurement implemented under four different conditions. A first angle torque is the initial angle torque. Following the measurement of the initial angle torque, a second angle torque was obtained after low-temperature plasma sterilization was repeated 100 times using hydrogen peroxide, wherein STERRAD NX produced by Johnson & Johnson was used as sterilizer. A third angle torque was obtained after the same sterilization was repeated another 100 times (making the number of times repeated 200). A fourth angle torque was obtained after the same sterilization was repeated another 100 times (now making the number of times repeated 300).

Where PTFE powder obtained by emulsion polymerization was used as lubricant, the angle torque after sterilization was implemented 100 times was found to have increased. Therefore, measurements of the angle torque after sterilization was implemented 200 times and 300 times, respectively, were conducted only with the endoscope using PTFE powder obtained by pyrolysis as lubricant.

Five different angle torques were measured: torque required to bend the angle section upwards by 90° (UP90), torque required to bend the angle section upwards by 130° (UP130), torque required to bend the angle section upwards by 180° (UP180), torque required to bend the angle section downwards (opposite direction to upwards) by 90° (DOWN90), and torque required to bend the angle section downwards by 180° (DOWN180).

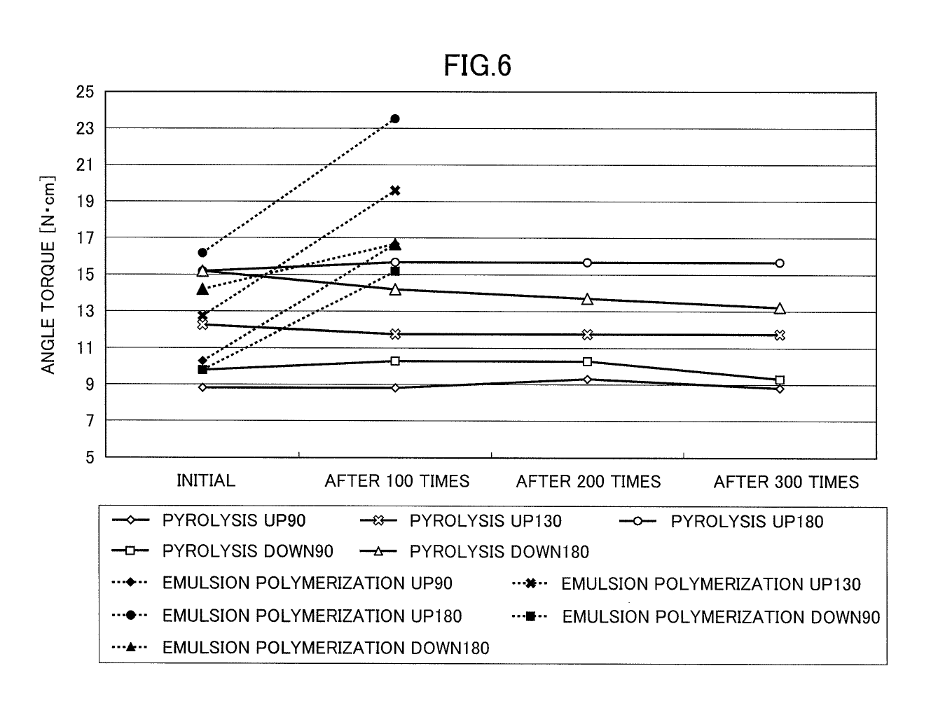

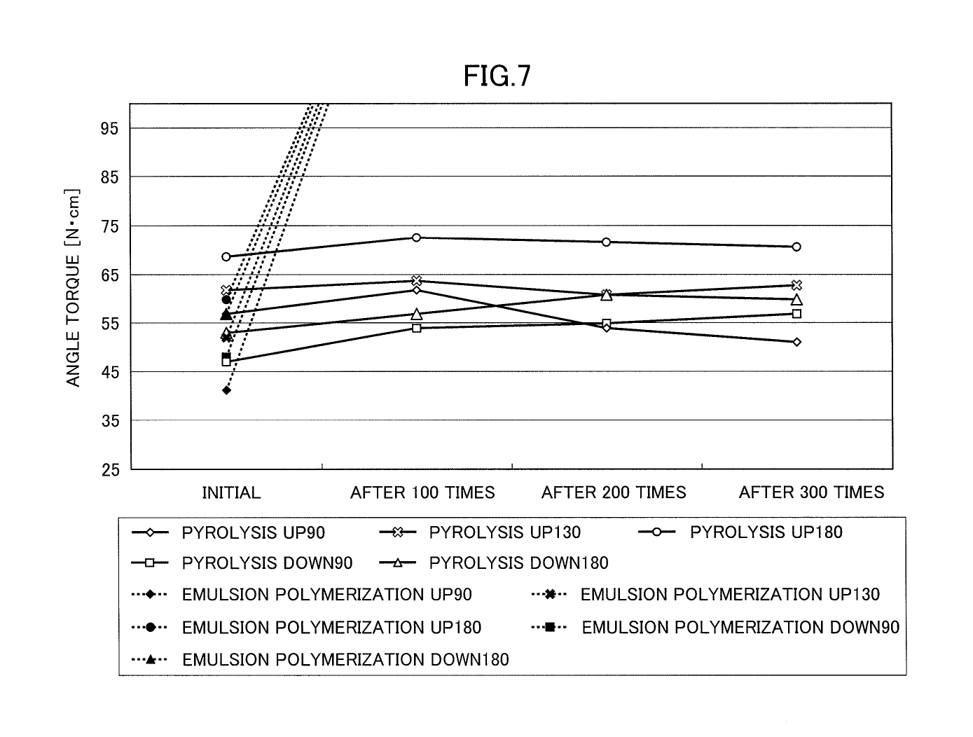

These angle torques were measured under two conditions: with the flexible section of the insertion section of the endoscope straightened (which may also be referred to as straight below), and with the flexible section of the insertion section of the endoscope formed into the shape of a loop (which may also be referred to as loop below). FIG. 6 illustrates the measurements obtained with the flexible section straightened; FIG. 7 illustrates the measurements obtained with the flexible section in the shape of a loop.

As illustrated in FIGS. 6 and 7, the endoscope where PTFE powder obtained by pyrolysis was used as lubricant for the wires exhibited only a small change in angle torque required for bending after the low-temperature plasma sterilization using hydrogen peroxide was performed 100 times, after the same sterilization was performed another 100 times (making the number of times repeated 200), and after the same sterilization was further performed another 100 times (making the number of times repeated 300), and this applies regardless of the angle of the angle section and whether the flexible section is in a straightened form or in the shape of a loop.

Further, the angle torque required for bending is smaller with most of the angles of the angle section than in the endoscope using PTFE powder obtained by emulsion polymerization.

By contrast, not only is a great angle torque for bending required in the endoscope using PTFE powder obtained by emulsion polymerization as described above, but the angle torque required for bending increases dramatically in such endoscope after low-temperature plasma sterilization using hydrogen peroxide.

In order to find the reasons therefor, the present inventors observed the particles of the PTFE powder. They found then that the PTFE powder obtained by emulsion polymerization has characteristics that the initial particles thereof are crushed and broken into yet finer particles by bending operation. The particles of PTFE powder obtained by emulsion polymerization are reduced by the crushing and breaking to such small dimensions that the lubricating effects to be produced by a solid lubricant against localized forces generated by sliding of the wires 68 over the angle section 26 (e.g., rings 60) as described above decrease, which is deemed to be a cause of insufficient lubricity.

Further, low-temperature plasma sterilization using hydrogen peroxide causes PTFE powder obtained by emulsion polymerization to re-agglomerate inappropriately and its properties to change. The re-agglomeration and change in properties are considered in turn to cause degradation of performance occurring where the PTFE powder obtained by emulsion polymerization is used as lubricant for the wire for bending the angle section.

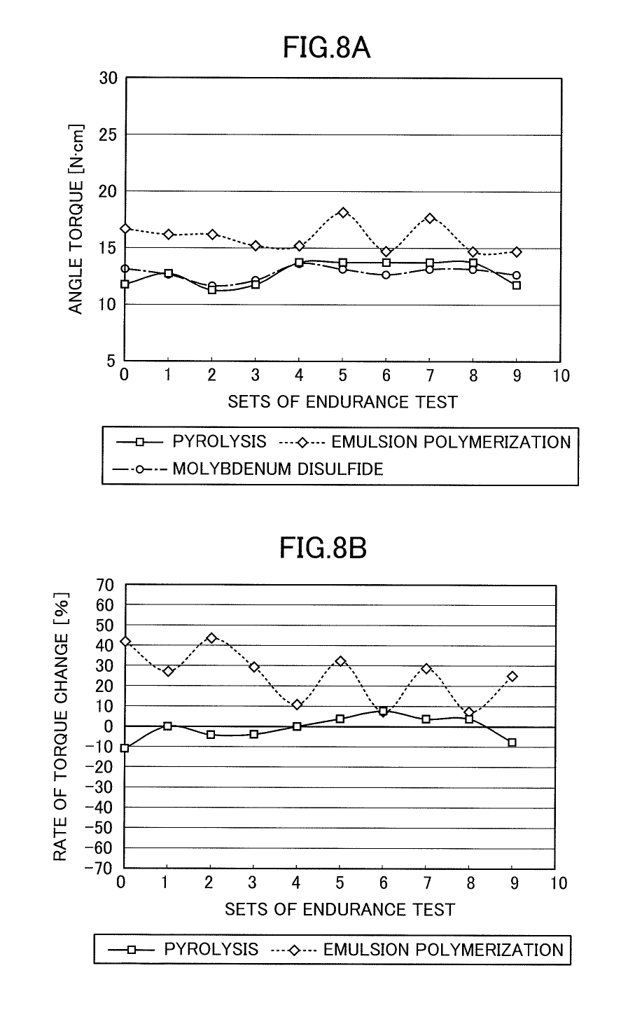

FIG. 8A illustrates the results of measurement of angle torques [N·cm] obtained after an endurance test in which a given operation of the angle section was repeated. The measurements were conducted with an endoscope using molybdenum disulfide powder as lubricant and endoscopes using the above-mentioned two kinds of PTFE powder as lubricant. The angle torque herein is a torque required for bending the above-mentioned straightened angle section downwards by 90°.

The endurance test was conducted in a total of 9 sets; the angle torque was measured after each set of the endurance test. The 9 sets of endurance test are approximately equivalent to frequent use of an endoscope for three years.

In relation to the above results of measurements, FIG. 8B illustrates a rate of torque change [%] measured after the endurance test in an endoscope using PTEF obtained by pyrolysis as lubricant and an endoscope using PTEF obtained by emulsion polymerization as lubricant.

As illustrated in FIG. 8A, use of PTFE powder obtained by pyrolysis as lubricant enables the angle section of the endoscope to be bent with a pull strength that is equivalent to or less than that required when molybdenum disulfide powder, which is conventionally used as lubricant, is used as lubricant for the wires, even at the start or after the endurance test is repeatedly performed. By contrast, where the endoscope uses PTFE powder obtained by emulsion polymerization as lubricant, a great angle torque is required for bending the angle section initially and after the endurance test is performed, as compared with the torques required in the above two kinds of endoscopes.

As illustrated in FIG. 8B, the angle torque is consistent in the endoscope using PTFE powder obtained by pyrolysis as lubricant even after the endurance test is repeatedly performed. Where the endoscope uses PTFE powder obtained by emulsion polymerization as lubricant, on the other hand, the angle torque changes greatly and, hence, the operation is inconsistent after the endurance test is performed repeatedly (i.e., through repeated use).

In other words, the present invention, wherein PTFE powder obtained by pyrolysis is used as lubricant applied to the wires for bending the angle section, permits the use of low-temperature plasma sterilization using hydrogen peroxide. Moreover, not only is the change in angle torque small through frequent and continued use of the endoscope, but the lubricant degrades little through cleaning, disinfection, and sterilization performed after each use of the endoscope. Further, the present invention provides an easy-to-operate endoscope permitting a high level of sterilization and enabling the angle section to be bent with a consistently small force over an extended period of time.

Various kinds of PTFE powder obtained by pyrolysis available on the market can also be used suitably for the purpose herein described.

The diameter of PTFE powder particles obtained by pyrolysis (“obtained by pyrolysis” is omitted hereinafter) used as lubricant for the wires 68 in the endoscope 10 of the invention is not specifically limited and preferably 5 to 50 μm.

Each individual line composing the twisted wires of the wires 68 described later typically has a diameter of about 50 μm. Therefore, PTFE powder having a particle diameter of 5 to 50 μm easily and stably adheres between the lines of the wires 68 and thus produces desirable results such as a long-lasting good lubricity.

To ensure a good lubricity, an appropriate amount of PTFE powder needs to lie over the surfaces of the wires 68 (between the wires 68 and the contact surface).

When the amount of PTFE powder is more than is appropriate, PTFE provides resistance and reduces lubricity over the wires 68. Lubricity is liable to degrade especially in a semi-closed space such as the coils 72 through which the wires 68 are passed.

Considering the above, the amount of PTFE powder to be adhered to the wires 68 is preferably in a range of 0.1 to 2 μg/cm and, in particular, 0.2 to 1 μg/cm.

The amount of PTFE powder in the above range can yield desirable results in that it can fill the semi-closed space defined by the coils 72 through which the wires 68 are passed to an appropriate degree and maintain a good lubricity for a long time.

In the endoscope 10, the wires 68 for bending the angle section 26 may be single lines and preferably composed of twisted wires, as described above. Preferably, the PTFE powder adheres not only to the surfaces of the wires 68 but permeates between the lines of the twisted wires of the wires 68.

PTFE powder weakly adheres to materials such as metals so that a sufficient amount of adhesion thereof to the wires 68 may not be secured or maintained in some cases. Where, on the other hand, the wires 68 are composed of twisted wires, and PTFE powder permeates between the lines of the twisted wires, an appropriate amount of PTFE powder can remain adhered to the wires 68 over an extended period of time.

PTFE powder may be adhered to the wires 68 by any method as appropriate including but not limited to known methods whereby powder is caused to adhere to linear material.

Examples of methods that may be used include one whereby PTFE powder is directly applied to the wires 68; one whereby PTFE powder is dispersed in a volatile solvent to prepare a coating material, which is applied onto the wires 68 by, for example, spraying or using a brush; and one using a tank filled with PTFE powder in which the wires 68 are immersed.

While the endoscope of the invention has been described in detail above, the invention is by no means limited to the above embodiments, and various improvements and modifications may of course be made without departing from the spirit of the present invention.

### INDUSTRIAL APPLICABILITY

The present invention may be used suitably for endoscopes employed for various diagnoses and examinations.

What is claimed is:

1. An endoscope comprising an angle section close to a distal end of an insertion section,
the endoscope further comprising a bending operation section for bending the angle section and wires for connecting the bending operation section and the angle section, the wires being drawn by operation of the bending operation section to bend the angle section, the angle section including substantially cylindrical tubular members connected in a longitudinal direction of the insertion section,
the wires having adhered on surfaces thereof polytetrafluoroethylene powder produced by pyrolysis as lubricant.

2. The endoscope according to claim 1, wherein the tubular members each have a projection formed by forming two cuts in a peripheral surface thereof arranged in a direction in which the insertion section extends and pressing a portion between the two cuts so as to bulge on an inside of each of the tubular members, and
wherein the wires are passed through the projections.

3. The endoscope according to claim 1, wherein the tubular members have an inner diameter of 5 mm or less.

4. The endoscope according to claim 1, wherein the polytetrafluoroethylene powder has a particle diameter of 5 to 50 μm.

5. The endoscope according to claim 1, wherein the polytetrafluoroethylene powder adheres to the wires in an amount of 0.1 to 2 μg/cm.

6. The endoscope according to claim 1, wherein the wires are twisted wires.

7. The endoscope according to claim 6, wherein the polytetrafluoroethylene powder permeates between lines constituting the twisted wires.

8. The endoscope according to claim 1, which is an endoscope for bronchi.
